(19) United States
(12) Patent Application Publication
(10) Pub. No.: US 2007/0037845 A1
(43) Pub. Date: Feb. 15, 2007

(54) Peptido-mimetic compounds containing rgd sequence useful as integrin inhibitors, and intermediates thereof

(75) Inventors: Carlo Scolastico, Milan (IT); Leonardo Pierpaolo Manzoni, Milan (IT); Matteo Giuseppe Colombo, Milan (IT); Marcello Di Giacomo, Milan (IT)

Correspondence Address: NIXON & VANDERHYE, PC, 901 NORTH GLEBE ROAD, 11TH FLOOR, ARLINGTON, VA, 22203, US

(73) Assignee: Universita' Degli Studi Di Milano, Milan (IT)

(21) Appl. No.: 10/563,288

(22) PCT Filed: Jul. 5, 2004
(86) PCT No.: PCT/IB04/02204

§ 371 Date: Mar. 22, 2006

(30) Foreign Application Priority Data
Jul. 18, 2003 (IT) MI2003A001476

(51) Int. Cl. A61K 31/4745; C07D 455/02

(52) U.S. Cl. 514/306; 546/138

(57) ABSTRACT

The subject of the present invention are cyclic compounds, in particular having azabicycloalkane structures of the general formula (I) 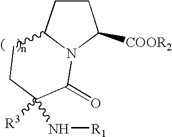 a process for their preparation, and their use as intermediates in the synthesis of biologically active peptidomimetic compounds containing the sequence RGD (Arg-Gly-Asp).

### SUBJECT OF THE INVENTION

Forming the subject of the present invention are cyclic compounds, in particular having an azabicycloalkane structure, a process for their preparation, and their use as intermediates in the synthesis of biologically active peptidomimetic compounds containing the sequence RGD (Arg-Gly-Asp).

### STATE OF THE ART

A large number of physiological processes involve biologically active peptides through their interactions with receptors and enzymes. Hence, for quite some time now much thought has been given to the development of peptide structures with high biological activity to be used as potential drugs for the treatment of several pathological conditions. However, peptides cannot be considered ideal drugs due to their poor metabolic stability, the high speed of excretion, and the low selectivity generally shown towards specific receptors. Studies have consequently been directed towards the design of analogues of peptides that are able to mimic the action of the corresponding natural peptides at a receptor level. Compounds with the aforesaid characteristics are commonly designated by the term “peptidomimetic”. For example, as described in U.S. Pat. No. 6,451,972, there have been studied peptidomimetic compounds containing a sequence RGD (Arg-Gly-Asp) and characterized by an azabicycloalkane structure, which show activity as inhibitors of cell adhesion mediated by αvβ3 integrines. Thanks to this biological activity, the aforesaid compounds are described as useful therapeutic agents in the treatment of pathological conditions due to altered angiogenesis, for example tumoral diseases.

One of the difficulties that have been noted in the use of biologically active peptides as possible drugs relates to the fact that peptide molecules can assume a wide range of conformations, which are not all equivalent and in particular are not all capable of interacting, for example with the receptors, in an equivalent way.

Also in the course of studies on peptidomimetic compounds, there has been noted a conformational freedom, which sometime is too high and has led, in some cases, to the loss of biological activity and to the reduction in selectivity and in the affinity of the peptidomimetic compound in regard to the receptor.

### OBJECTS OF THE INVENTION

An object of the present invention is to make available compounds having an azabicycloalkane structure that will be useful intermediates in the synthesis of peptidomimetic compounds with biological activity.

A further object of the present invention is to make available a process for the preparation of said compounds having an azabicycloalkane structure.

Yet another object of the present invention is to provide a process for the synthesis of peptidomimetic compounds that will envisage the use of said azabicycloalkanes.

Yet a further object of the present invention is to make available peptidomimetic compounds comprising the azabicycloalkane structure and the RGD sequence which will be constrained from the conformational point of view.

A further object of the present invention is to make available peptidomimetic compounds that will present biological activity as angiogenesis inhibitors and that may be used as drugs for example with antitumoral activity.

Finally, another object of the invention is to make available peptidomimetic compounds that may be used as vehicles for the transport of molecules with pharmacological activity, enabling easy releasing thereof in situ.

### DESCRIPTION

These and yet other purposes, as well as the corresponding advantages that will emerge more clearly from the following description, are achieved by compounds having the following general formula: 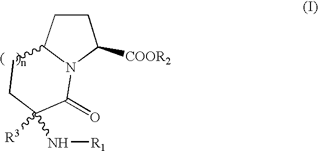
where:
R₁ is chosen from hydrogen, a lower alkyl, and a suitable protective group of the amine;
R₂ is chosen between hydrogen, and a suitable protective group of the carboxyl;
R₃ is chosen from benzyl, substituted benzyl, allyl, hydroxypropyl, hydroxyethyl, lower alkyl;
n is a number chosen from 0, 1, 2;
   including the salts, the racemates, the individual enantiomeric forms, the individual diastereoisomeric forms, or their mixtures.

In the formula indicated above, and in general in all the formulae that will be indicated, the sign

[symbol]

designates a bond that can be above or below the plane of the page.

In general, by “suitable protective group of the amine” or “suitable protective group of the carboxyl” is meant a protective group as given in the following examples, as is known to the skilled person and as appears from the relevant technical literature and commercial catalogues.

In particular, examples of appropriate protective groups are alkyl or benzyl esters.

By “lower alkyl group” is meant a C₁-C₄ alkyl group, for example methyl, ethyl, propyl, butyl and all the possible isomers, but also higher alkyls are possible provided that they are compatible with the reaction conditions.

The compounds of formula (I) have an azabicycloalkane structure and are characterized by the presence of a substituent on the carbon atom in position 3. This substituent is capable of reducing the conformational degrees of the molecule and, if for example it is of an alkyl nature, can moreover give characteristics of greater hydrophobicity to the molecule, as well as, if it is provided with an appropriate functional group, for example hydroxyl, being able to act as “binding agent” for different fragments or 5 molecules provided, for example, with pharmacological activity. According to the present invention, the preferred compounds of the general formula (I) are the following:
n is chosen equal to 1, and R₃ is chosen as a benzyl;
n is chosen equal to 1, and R₃ is chosen as an allyl;
n is chosen equal to 2, and R₃ is chosen as a benzyl;
n is chosen equal to 2, and R₃ is chosen as an allyl;
n is chosen equal to 2, and R₃ is chosen as a methyl.

The subject of the present invention is a process for the preparation of compounds having the general formula (I). In particular, with reference to FIG. 1

[image]

which shows a generic scheme of synthesis of compounds of formula (I), the process comprises the following steps:

formation, in suitable reaction conditions, of the carbanion in position 3, starting from the compound (Ia) or from one of its suitable derivatives; and
alkylation of the carbanion to obtain the compound of the general formula (I).

In the case of the scheme of FIG. 1, the substituents are defined as follows:
R₁ is chosen from hydrogen, lower alkyl, suitable protective group of the amine;
R₂ is chosen between hydrogen, and a suitable protective group of the carboxyl;
R₃ is chosen from benzyl, substituted benzyl, allyl, hydroxypropyl, hydroxyethyl, and lower alkyl;
n is a number chosen from 0, 1, 2;
   including the salts, the racemates, the individual enantiomeric forms, the individual diastereoisomeric forms, or their mixtures.

In particular, just by way of example, FIG. 1a

[image]

is a schematic representation of the process for preparation of compounds of the general formula (I), where R₁ is the carbobenzyloxy (Cbz) group, whilst R₂, n and R₃ are defined as above. In this case, the process envisages the following steps:

reaction of chemoselective deprotection of the nitrogen atom in position 3 of the compound of the general formula (Ib), and formation of the corresponding imine, of the general formula (Ic);
deprotonation in position 3 of the compound of the general formula (Ic) with formation of the corresponding enolate, reaction of alkylation of said enolate, and reaction of reduction of the double iminic bond to obtain the compound of the general formula (Id).

In this case, the process for the preparation of compounds of the general formula (I) and, in the case of the specific example, the process for the preparation of compounds of the general formula (Id), envisages the reaction of stereoselective alkylation of the enolate of the compounds of formula (Ic). The starting products used in the process described above are prepared according to methods already known in the literature, for example as described in EP 1 077 218, Angiolini, M.; Araneo, S.; Belvisi, L.; Cesarotti, E.; Checchia, A.; Crippa, L.; Manzoni, L.; Scolastico, C. Eur. J. Org. Chem. 2000, 2571-2581; Manzoni, L.; Colombo, M.; May, E.; Scolastico, C. Tetrahedron 2001, 57, 249.

FIGS. 2 and 3 show, purely by way of example, the scheme of the process according to FIG. 1a, where the substituent R₂ is chosen as tBu. In this case, the reaction conditions are given in detail for the individual passages performed and the products obtained according to the type of alkylating agent used. FIG. 2 refers to the process for obtaining the “trans” product

[image]

| | | | | | | Ratio |
|---|---|---|---|---|---|---|
| | | | | | | (3R)/ |
| n | Base | T (° C.) | R3 | Product | Yield | (3S) |
| 1 | LiHMDS | −78→rt | —CH₂Ph | 3, 4 | 56% | 92:8 |
| 1 | LiHMDS | −50 | —CH₂Ph | 3, 4 | 89% | 90:10 |
| 1 | LiHMDS + | −78→rt | —CH₂Ph | 3, 4 | 43% | 5:95 |
| | Mg⁺⁺ | | | | | |
| 1 | LiHMDS + | −50→−20 | —CH₂Ph | 3, 4 | 43% | >2:98 |
| | Mg⁺⁺ | | | | | |
| 1 | LiHMDS | −50 | —CH₂CH═CH₂ | 5, 6 | 90% | 84:16 |
| 1 | LiHMDS + | −78→rt | —CH₂CH═CH₂ | 5, 6 | 55% | 7:93 |
| | Mg⁺⁺ | | | | | |
| 1 | LiHMDS + | −50→−20 | —CH₂CH═CH₂ | 5, 6 | 45% | >2:98 |
| | Mg⁺⁺ | | | | | |
| 2 | LiHMDS | −50 | —CH₂Ph | 7, 8 | 82% | 40:60 |
| 2 | LiHMDS + | −78→rt | —CH₂Ph | 7, 8 | 68% | >2:98 |
| | Mg⁺⁺ | | | | | |
| 2 | NaHMDS | −78→rt | —CH₂Ph | 7, 8 | 81% | 10:90 |
| 2 | NaHMDS + | −78→rt | —CH₂Ph | 7, 8 | 59% | 9:91 |
| | DMPU | | | | | |
| 2 | LiHMDS | −50 | —CH₂CH═CH₂ | 9, 10 | 67% | 55:45 |
| 2 | LiHMDS + | −78→rt | —CH₂CH═CH₂ | 9, 10 | 40% | 6:94 |
| | Mg⁺⁺ | | | | | |
| 2 | LiHMDS | −78→rt | —CH₃ | 11, 12 | 69% | 78:22 |

whilst FIG. 3 refers to the process for obtaining the “cis” product.

[image]

| | | | | | | Ratio |
|---|---|---|---|---|---|---|
| | | | | | | (3/R)/ |
| n | Base | T (° C.) | R3 | Product | Yield | (3S) |
| 1 | LiHMDS + | −78→rt | —CH₂Ph | 13, 14 | 72% | 9:91 |
| | Mg⁺⁺ | | | | | |
| 1 | NaHMDS | −78→rt | —CH₂Ph | 13, 14 | 81% | 23:77 |
| 1 | KHMDS | −78→rt | —CH₂Ph | 13, 14 | 58% | 7:93 |
| 1 | KHMDS + | −78→rt | —CH₂Ph | 13, 14 | 37% | >2:98 |
| | DMPU | | | | | |
| 1 | LiHMDS | −78→rt | —CH₂CH═CH₂ | 15, 16 | 63% | 10:90 |
| 1 | LiHMDS + | −78→rt | —CH₂CH═CH₂ | 15, 16 | 42% | >2:98 |
| | Mg⁺⁺ | | | | | |
| 2 | LiHMDS | −78→rt | —CH₂Ph | 17, 18 | 65% | 55:45 |
| 2 | LiHMDS + | −78→rt | —CH₂Ph | 17, 18 | 70% | 65:35 |
| | Mg⁺⁺ | | | | | |
| 2 | LiHMDS | −78→rt | —CH₂CH═CH₂ | 19, 20 | 58% | 53:47 |
| 2 | LiHMDS + | −78→rt | —CH₂CH═CH₂ | 19, 20 | 55% | 60:40 |
| | Mg⁺⁺ | | | | | |

The synthesis of the products numbered from 3 to 20 and given in FIGS. 2 and 3 was obtained according to what is already represented schematically in FIG. 1. In particular, the starting bicyclic lactams were chemoselectively deprotected by means of hydrogenation at atmospheric pressure using Pd/C. The amines obtained were converted into the corresponding Shiff bases for treatment with benzaldehyde in the presence of triethylamine and MgSO₄. Stereoselective alkylation of the enolate of the amide of the Shiff base leads to the corresponding alkyl derivatives, which were subsequently reduced with NaBH₄ to yield the lactams 3-20.

As appears from the literature, the alkylation reactions depend upon a series of factors, such as solvent, counter-ion, and temperature, which are all parameters that influence enormously both the yields and the stereochemical course of the reaction.

The reaction conditions, the yields and the stereochemistry of the reaction of alkylation in the position C3 are, as has already been said, illustrated in the tables (FIGS. 2 and 3). The stereochemistry of the stereocentres that are formed in the course of the reaction was determined by means of NOE experiments and x-rays and will be given in detail in the examples corresponding to the ensuing experimental part.

Once again with reference to the compounds of the general formula (I), in the case where the substituent R₃ is chosen as an allyl, it is possible to perform a further conversion of the allyl substituent in general into a hydroxyl group, for example by means of a hydroboration reaction. In particular, it is possible to obtain hydroxypropyl or hydroxyethyl groups. In the first case, the hydroxypropyl group is obtained by a reaction of hydroboration and decomposition, for example with alkaline H₂O₂, whereas in the second case the hydroxyethyl group is obtained, for example, by reductive ozonolysis of the double bond.

FIG. 4 presents,

[image]

by way of example, a complete scheme of the reaction conditions for conversion of the allyl group in position 3 into the hydroxyethyl group starting from the compound 5, where, with respect to the general formula (I), the substituents are selected as follows: n is chosen equal to 1, R₃ is chosen as an allyl, R₂ is chosen as tBu, and R₁ is chosen as CH₂Ph.

Once again in the case where the group R₃ is chosen as an allyl, it is possible to carry out conversion thereof into other derivatives according to the compatibility between the general structure of the molecule and the reaction conditions required for conversion.

The compounds of formula (I) are used to advantage as intermediates in the synthesis of peptidomimetic compounds with reduced conformational freedom.

The compounds of the general formula (I), according to the present invention, are used as intermediates in the synthesis of biologically active peptidomimetic compounds, in particular in the synthesis of cyclic peptidomimetic compounds comprising the sequence RGD (Arg-Gly-Asp) (Arginine, Glycine, Aspartic acid) of the general formula (II), as given hereinafter: 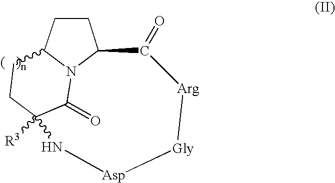
where:
R₃ is chosen from benzyl, substituted benzyl, allyl, hydroxypropyl, hydroxyethyl, lower alkyl;
n is a number chosen from 0, 1, 2;
   including the salts, the racemates, the individual enantiomeric forms, the individual diastereoisomeric forms, or their mixtures.

In the formula indicated above, and in general in all the formulae that will be indicated, the sign

[symbol]

indicates a bond that can be above or below the plane of the page.

By “lower alkyl group” (lower alkyl) is meant a C₁-C₄ alkyl group, for example a methyl, ethyl, propyl, butyl, and all the possible isomers, but also higher alkyls are possible, provided that they are compatible with the reaction conditions.

The compounds of formula (II) are synthesised, starting from the compounds of formula (I), according to a general process, which comprises the following steps:
reaction of chemoselective deprotection of the carboxylic group of the compound of the general formula (I), and condensation with the Arg-Gly dipeptide appropriately protected and previously prepared;
reaction of chemoselective protection of the amine group of the azabicycloalkane by means of catalytic hydrogenation, and subsequent condensation with appropriately protected aspartic acid;
conversion of the methyl ester of glycine into benzyl esters by means of a transesterification reaction, followed by simultaneous removal of the protective group of glycine and of the amine group of aspartic acid by means of catalytic hydrogenation; and
intramolecular cyclization mediated by condensing agents, and subsequent deprotection of the protective groups of the side chains of the amino acids.

In particular, FIG. 5 provides an example of process for the preparation of a peptidomimetic compound comprising the RGD sequence according to the present invention of formula (II), where R₃ is chosen as CH₂Ph and n is chosen equal to 1, to obtain the compound designated by 28.

[image]

[image]

72% (on 2 passages); viii. CF₃COOH, scavengers; ix. HCl, 96% (on 2 passages).

In the aforesaid process, the starting point is the compound of formula (I), where R₃ is chosen as CH₂Ph, n is chosen equal to 1, R₁ is chosen as CH₂Ph, and R₂ is chosen as tBu (compound 3). Once again appearing in FIG. 5 are the various reagents used in the various steps of the process, as well as the corresponding reaction conditions. In this case, the diagram of synthesis is exemplified for just one diastereoisomer, but it remains understood that, in a similar way, the process extends to the totality of the compounds forming the subject of the present invention.

Once again according to the present invention, the preferred compounds chosen between those of the general formula (II) are the following:
a) when n is chosen equal to 1, and R₃ is chosen as a benzyl
b) when n is chosen equal to 2, and R₃ is chosen as a benzyl.

FIG. 6 illustrates the most representative compounds of the general formula (II).

[image]

[image]

In particular, according to the present invention, the most significant compound, given hereinafter, has the formula designated by number 26, again with reference to FIG. 6 mentioned above.

The compounds of the general formula (II) according to the present invention show biological activity as inhibitors of integrines, and in particular are selective inhibitors for αvβ3 and αvβ5 integrines. The compounds of formula (II) will be used as drugs for inhibiting angiogenesis, for example in the treatment of pathological conditions of a tumoral origin, as in the case of metastasized tumoral processes, retinopathies, acute renal damage and osteoporosis.

With reference to the activity of the compounds of the general formula (II) in regard to αvβ3 and αvβ5 integrines, FIG. 7 gives the results corresponding to the biological tests carried out for evaluating the binding properties of the aforesaid compounds in regard to the aforesaid αvβ3 and αvβ5 receptors. The tests have been conducted according to the modalities of the known art, in particular according to what is described in EP 1077218, for example on pages 10-14.

[image]

Inhibition of the binding of [¹²⁵I]-echistatine on the αᵥβ₃ and αvβ5 receptors. The IC₅₀ values are calculated as the concentration of compounds required for the inhibition of 50% of the binding of the echistatine as evaluated by the program Allfit. All the values are the average (±standard deviation) of triplicate determinations.

The presence of an aryl/alkyl substituent in position 3 on the compounds of the general formula (II) according to the present invention gives to the peptidomimetic compound a greater conformational rigidity thanks also to the steric interactions between the substituent and the cyclic structure, which can flavour the interaction between the compound and the receptor. The compounds according to the present invention, when used as drugs, may thus more easily reach the tissues that overexpress certain receptors (for example epithelial cells involved in vascular growth) and thus express their pharmacological activity.

The compounds according to the present invention can hence be viewed as conformationally constrained “scaffolds”, with the potentiality of replicating the geometry of the skeleton and of the side chains of a dipeptide residue in the active site. The sequence of amino acids selected and inserted in the structure of the compounds in question can be used as a conformationally constrained entity which mimics segments of natural peptides. Alternatively, the functionalized side chains can be used as site for the introduction of groups that are important from the pharmacological standpoint, for example for increasing proteine-proteine or protein-receptor interactions.

Another possible application for the compounds of the general formula (II) is their use as “reverse-turn” inducers and, as has already been said, as “scaffolds” for the synthesis of biologically active compounds.

Once again according to the present invention, the compounds of formula (II) are also used as mediators for the transport and release of drugs. For example, since they themselves show activity as angiogenesis inhibitors, they may to advantage be conjugated to a compound provided with pharmacological activity of the cytotoxic type so as to enable simultaneous administration of two different active principles (in the case exemplified, a cytotoxic active principle and an anti-angiogenesis active principle). The additional compound can be bound to the compound of formula (II) in a conventional way, for example through reactive groups available for the formation of a chemical bond. The release of the additional compound with pharmacological activity will take place in situ in physiological conditions. In particular, in the case of the compounds of formula (II) defined as above, the most suitable group for the further reaction with an additional compound is R₃ chosen as a hydroxyethyl or a hydroxypropyl.

In some cases, also the compound of formula (I) can be used, via the R₃ group appropriately selected, for example as hydroxyethyl or hydroxypropyl, for association to a pharmacologically active compound, prior to its conversion into a peptidomimetic compound of the general formula (II). In this case, it is, however, necessary for the reaction scheme that involves the intermediate of formula (I) to yield the compound of formula (II) to be compatible with the presence of the additional pharmacologically active compound bound to the principal structure via the substituent R₃.

Forming the subject of the present invention are the pharmaceutical compositions that comprise an effective dose, from the therapeutic standpoint and/or from the prophylactic standpoint, of at least one compound of formula (II) in a mixture with vehicles and/or excipients that are acceptable from the pharmaceutical point of view.

The pharmaceutical compositions referred to above are used as inhibitors of integrines, and in particular selective inhibitors for αvβ3 and αvβ5 integrines. The pharmaceutical compositions comprising at least one compound of formula (II) are then used as drugs for inhibiting angiogenesis, for example in the treatment of pathological conditions of a tumoral origin, as in the case of metastasized tumoral processes, retinopathies, acute renal damage and osteoporosis.

The present invention will be described in detail with the aid of the examples given hereinafter, which are provided purely by way of explanatory and non-limiting example of the field of protection of the invention.

General remarks: The ¹H- and ¹³C-NMR spectra were recorded in CDCl₃ (or D₂O) as indicated, at 200 (or 300, 400) and 50.3 (or 75.4) MHz, respectively. The values of chemical shift are indicated in ppm, and coupling constants in Hz.—The optical rotary powers were measured with a Perkin-Elmer polarimeter model 241.—Thin-layer chromatography (TLC) was performed using F-254 Merck plates. Flash chromatography was performed using Macherey-Nagel 60, 230-400 mesh silica gel. The solvents were dehydrated in accordance with standard procedures, and the reactions requiring anhydrous conditions were conducted in a nitrogen or argon atmosphere. The solutions containing the end products were dehydrated using Na₂SO₄, filtered, and concentrated at reduced pressure using a rotary evaporator.

By “lactam” is meant the compound of the general formula (I) in all its forms of possible substitution; by “pseudopeptide” is meant a compound of the general formula (II) in all its forms of possible substitution.

### EXAMPLE 1

General Procedure A: Preparation of the Imine.

A solution of lactams protected as carbobenzyloxy derivatives (1.07 mmol) (compound (Ia) where R₁ is chosen as Cbz) in MeOH (11 ml) containing a catalytic quantity of 10% Pd/C was stirred overnight in a hydrogen atmosphere. The catalyst was removed by filtration on Celite and washed with MeOH. The solvent was evaporated at reduced pressure. The crude product was dissolved in anhydrous CH₂Cl₂ (11 ml) and anhydrous TEA (299 μl, 2.14 mmol); there were then added MgSO₄ (64 mg) and benzaldehyde, previously distilled. After 24 hours at room temperature the mixture was filtrated on Celite and washed with CH₂Cl₂. The solvent was removed at reduced pressure to the initial amount, and then the same amount of hexane was added. The organic solution, washed with saturated NaHCO₃ (2×20 ml), water (2×20 ml) and brine (2×20 ml), was then dehydrated on Na₂SO₄ and evaporated at reduced pressure. The crude product (90-95% in 2 passages, white solid) was used without further purification.

General procedure B: Alkylation of the Imine

To a solution of imine (0.2 mmol) in anhydrous THF (2 ml) in an argon atmosphere, cooled to −78° C., there was added the base (0.3 mmol), and the temperature was adjusted according to the indications appearing in the tables of FIGS. 2 and 3. After 20 minutes allyl, benzyl bromide or iodomethane (0.4 mmol) were added, and the solution was stirred 3-5 hours. Water (2 ml) was added, and the mixture was extracted with AcOEt (3×2 ml). The reunited organic phases were dehydrated on Na₂SO₄ and evaporated at reduced pressure. To the crude product dissolved in MeOH (4 ml) there was added NaBH₄ (2 mmol) in small portions. The solvent was evaporated at reduced pressure, and the crude product was purified by flash chromatography (Hexane/AcOEt 7:3).

General Procedure C: Alkylation of the Imine in the Presence of DMPU

To a solution of imine (0.2 mmol) in anhydrous THF (2 ml) and DMPU (5 mmol) in an argon atmosphere, cooled to −78° C. there was added the base (0.3 mmol), and the temperature was adjusted according to what is set out in the tables of FIGS. 2 and 3. After 20 minutes allyl, benzyl bromide or iodomethane (0.4 mmol) were added, and the solution is stirred 3-5 hours. After the addition of water (2 ml), the mixture was extracted with AcOEt (3×2 ml). The reunited organic phases were dehydrated on Na₂SO₄ and evaporated at reduced pressure. To the crude product dissolved in MeOH (4 ml) there was added NaBH₄ (2 mmol) in small portions. After evaporation at reduced pressure the crude product was purified by flash chromatography (Hexane/AcOEt 7:3).

General procedure D: Alkylation of Imine in the Presence of a Chelating Salt

To the solution of imine (0.2 mmol) in anhydrous THF (2 ml) in an argon atmosphere, cooled to −78° C., there was added the base (0.3 mmol), and the temperature was adjusted as illustrated in the tables of FIGS. 2 and 3. After 20 minutes, there was added a Lewis acid (MgBr₂.Et₂O or SnCl₂) (0.6 mmol), and after another 20 minutes allyl, benzyl bromide or iodomethane (0.4 mmol) were added leaving the solution under stirring for 3-5 hours. There was added water (2 ml), and the mixture was extracted with AcOEt (3×2 ml). The reunited organic phases were dehydrated on Na₂SO₄ and evaporated at reduced pressure. To the crude product dissolved in MeOH (4 ml) there was added NaBH₄ (2 mmol) in small portions. The solvent was evaporated at reduced pressure, and the crude product purified by flash chromatography (Hexane/AcOEt 7:3).

Likewise, the compounds from 3 to 20 according to FIGS. 2 and 3 were prepared; the corresponding analytical data are given below.

Lactam 3: [α]_{D}²²=−107.1 (c=1.05, CHCl₃). ¹H NMR (300 MHz, CDCl₃): δ 0.51 (m, 1H), 1.03 (m, 1H) 1.49 (s, 9H, COOtBu), 1.61-2.2 (5H), 2.31 (m, 1H), 2.81, 3.26 (2 d, 2H, J=12.8 Hz, PhCH₂C), 3.60 (m, 1H, CHN), 3.74, 3.80 (2 d, 2H, J=11.6 Hz, PhCH₂NH), 4.41 (dd, 1H, J=8.6 Hz, J=8.6 Hz, CHCOOtBu), 7.19-7.40 (10H, Ph). ¹³C NMR (50.3 MHz, CDCl₃): δ 172.7, 172.0, 140.7, 137.4, 130.4, 128.8, 128.6, 128.3, 127.1, 126.9, 81.5, 62.6, 59.9, 59.7, 48.2, 47.2, 33.5, 29.3, 28.3, 28.2, 26.6. FAB⁺MS: calc. C₂₇H₃₄N₂O₃ 434.26, found 435 [M+1]⁺. Elem. anal. calc. C₂₇H₃₄N₂O₃: C 74.62, H 7.89, N 6.45; found C, 74.50; H, 7.98; N, 6.32.

Lactam 4: pf=104-106° C. [α]_{D}²²=−37.0 (c=1.00, CHCl₃). ¹H NMR (300 MHz, CDCl₃): δ 1.51 (s, 9H, COOtBu), 1.65-2.12 (7H), 2.26 (m, 1H), 2.98, 3.23 (2 d, 2H, J=13.1 Hz, PhCH₂C), 3.43 (m, 1H, CHN), 3.72, 3.84 (2 d, 2H, J=12.0 Hz, PhCH₂NH), 4.41 (dd, 1H, J=8.6 Hz, J=8.6 Hz, CHCOOtBu), 7.20-7.37 (10H, Ph). ¹³C NMR (50.3 MHz, CDCl₃): δ 171.9, 171.6, 137.03, 131.2, 128.5, 128.4, 128.2, 127.1, 126.6, 81.4, 61.0, 60.1, 59.5, 48.2, 44.7, 33.3, 30.5, 28.2, 28.1, 27.1. FAB⁺MS: calc. C₂₇H₃₄N₂O₃ 434.26, found 435 [M+1]⁺. Elem. anal. calc. C₂₇H₃₄N₂O₃: C, 74.62; H, 7.89; N, 6.45; found C, 74.77; H, 7.79; N, 6.35.

Lactam 5: pf=75-77° C. [α]_{D}²²=−71.8 (c=0.99, CHCl₃). ¹H NMR (300 MHz, CDCl₃): δ 1.47 (s, 9H, COOtBu), 1.50 (m, 1H), 1.79 (m, 1H), 1.88-2.19 (4H), 2.22-2.55 (4H), 3.68, 3.78 (2 d, 2H, J=11.7 Hz, PhCH₂NH), 3.74 (m, 1H, CHN), 4.40 (dd, 1H, J=8.6 Hz, J=8.6 Hz, CHCOOtBu), 5.10 (m, 2H, CH═CH₂), 5.87 (m, 1H, CH═CH₂), 7.16-7.43 (5H, Ph). ¹³C NMR (75.4 MHz, CDCl₃): δ 171.7, 133.4, 130.9, 128.7, 128.4, 127.1, 118.7, 111.1, 81.4, 61.6, 60.1, 59.1, 48.1, 45.3, 44.1, 33.2, 29.7, 29.2, 28.0, 26.5. FAB⁺MS: calc. C₂₃H₃₂N₂O₃ 384.24, found 385 [M+1]⁺. Elem. anal. calc. C₂₃H₃₂N₂O₃: C 71.84, H 8.39, N 7.29; found C, 71.99; H, 8.21; N, 7.36. Lactam 6: [α]_{D}²²=37.3 (c=1.00, CHCl₃). ¹H NMR (300 MHz, CDCl₃): δ 1.47 (s, 9H, COOtBu), 1.50 (m, 1H), 1.76 (m, 1H), 1.89-2.06 (3H), 2.18 (m, 1H), 2.26-2.43 (3H), 2.54 (m, 1H), 3.61 (m, 1H, CHN), 3.61, 3.70 (2 d, 2H, J=11.7 Hz, PhCH₂NH), 4.43 (dd, 1H, J=8.6 Hz, J=8.6 Hz, CHCOOtBu), 5.11 (m, 2H, CH═CH₂), 5.90 (m, 1H, CH═CH₂), 7.20-7.34 (5H, Ph). ¹³C NMR (75.4 MHz, CDCl₃): δ 171.9, 171.3, 140.4, 134.0, 129.1, 128.7, 128.3, 128.0, 126.9, 118.7, 81.3, 60.0, 59.7, 59.4, 51.1, 48.2, 45.3, 33.2, 30.4, 28.1, 28.0, 27.8. FAB⁺MS: calc. C₂₃H₃₂N₂O₃ 384.24, found 385 [M+1]⁺. Elem. anal. calc. C₂₃H₃₂N₂O₃: C, 71.84; H, 8.39; N, 7.29; found C 71.89, H 8.18, N 7.16.

Lactam 7: [α]_{D}²²=+36.4 (c=1.11, CHCl₃). ¹H NMR (300 MHz, CDCl₃): δ 1.44 (s, 9H, COOtBu), 1.49 (m, 3H), 1.58-1.72 (3H), 1.80-1.97 (2H), 2.12 (m, 1H), 2.29 (m, 1H), 2.92, 3.54 (2 d, 2H, J=14.1 Hz, PhCH₂C), 3.96, 4.04 (2 d, 2H, J=12.1 Hz, PhCH₂NH), 4.55 (dd, 1H, J=8.4 Hz, J=3.7 Hz, CHCOOtBu), 4.84 (m, 1H, CHN), 7.15-7.50 (10H, Ph). ¹³C NMR (50.3 MHz, CDCl₃): δ 174.5, 171.6, 141.0, 138.4, 131.5, 131.2, 129.0, 128.9, 128.7, 128.6, 128.5, 128.4, 128.3, 128.2, 128.0, 127.1, 126.3, 81.0, 64.0, 62.7, 57.6, 47.7, 40.3, 35.7, 33.1, 32.7, 29.9, 28.3, 26.9, 23.0. FAB⁺MS: calc. C₂₈H₃₆N₂O₃ 448.27, found 449 [M+1]⁺. Elem. anal. calc. C₂₈H₃₆N₂O₃: C 74.97, H 8.09, N 6.24; found C, 74.88; H, 7.99; N, 6.33.

Lactam 8: pf=113-114° C. [α]_{D}²²=−20.1 (c=1.06, CHCl₃). ¹H NMR (300 MHz, CDCl₃): δ 1.49 (s, 9H, COOtBu), 1.64-1.78 (3H), 1.78-1.96 (4H), 2.12 (m, 1H), 2.38 (m, 2H), 2.92, 3.13 (2 d, 2H, J=13.6 Hz, PhCH₂C), 3.61, 3.70 (2 d, 2H, J=12.5 Hz, PhCH₂NH), 4.14 (m, 1H, CHN), 4.55 (dd, 1H, J=8.3 Hz, J=2.0 Hz, CHCOOtBu), 7.17-7.43 (10H, Ph). ¹³C NMR (50.3 MHz, CDCl₃): δ 174.3, 172.1, 141.8, 136.4, 131.6, 128.3, 128.2, 127.9, 126.6, 81.2, 65.8, 62.3, 57.1, 48.1, 44.6, 34.4, 32.5, 32.2, 28.2, 26.5, 22.6. FAB⁺MS: calc. C₂₈H₃₆N₂O₃ 448.27, found 449 [M+1]⁺. Elem. anal. calc. C₂₈H₃₆N₂O₃: C, 74.97; H, 8.09; N, 6.24; found C, 75.18; H, 8.00; N, 6.13.

Lactam 9: [α]_{D}²²=+14.9 (c=1.04, CHCl₃). ¹H NMR (300 MHz, CDCl₃): δ 1.41 (s, 9H, COOtBu), 1.44-2.34 (10H, CH₂), 2.43, 2.87 (2 dd, 2H, J=14.4 Hz, J=7.3 Hz, CH₂—CH═CH₂), 3.73 (2 d, 2H, J=12.7 Hz, NH—CH₂-Ph), 4.49 (dd, 1H, J=8.3 Hz, J=4.4 Hz, CH—COOtBu), 4.79 (m, 1H, CO—N—CH), 5.16 (m, 2H, CH₂—CH═CH₂), 5.86 (m, 1H, CH₂—CH═CH₂), 7.20-7.40 (5H, Ph). ¹³C NMR (75.4 MHz, CDCl₃): δ 174.4, 171.4, 141.1, 134.5, 128.7, 128.3, 126.8, 118.7, 111.4, 80.7, 67.0, 62.8, 62.5, 58.5, 57.5, 47.1, 44.7, 40.3, 35.5, 33.1, 29.7, 28.0, 26.8, 22.7. FAB⁺MS: calc. C₂₄H₃₄N₂O₃ 398.26, found 399 [M+1]⁺. Elem. anal. calc. C₂₄H₃₄N₂O₃: C, 72.33; H, 8.60; N, 7.03; found C, 72.48; H, 8.41; N, 7.16.

Lactam 10: [α]_{D}²²=−54.0 (c=1.00, CHCl₃). ¹H NMR (300 MHz, CDCl₃): δ 1.45 (s, 9H, COOtBu), 1.63-1.98 (8H, CH₂), 2.12, 2.29 (2 m, CH₂), 2.49, 2.58 (2 m, 2H, CH₂—CH═CH₂), 3.68, 3.73 (2 d, 2H, J=11.6 Hz, NH—CH₂-Ph), 4.07 (m, 1H, CO—N—CH), 4.53 (dd, 1H, J=8.3 Hz, J=3.8 Hz, CH—COOtBu), 5.14 (m, 2H, CH₂—CH═CH₂), 5.88 (m, 1H, CH₂—CH═CH₂), 7.26, 7.42 (2 m, 5H, Ph). ¹³C NMR (50.3 MHz, CDCl₃): δ 171.8, 135.0, 128.6, 128.4, 128.3, 126.9, 118.9, 81.3, 63.1, 57.7, 48.2, 48.0, 35.2, 34.8, 32.8, 32.3, 29.9, 28.2, 26.5, 22.6. FAB⁺MS: calc. C₂₄H₃₄N₂O₃ 398.26, found 399 [M+1]⁺. Elem. anal. calc. C₂₄H₃₄N₂O₃: C, 72.33; H, 8.60; N, 7.03; found C 72.26, H 8.54, N 6.93.

Lactam 11: [α]_{D}²²=−22.1 (c=1.04, CHCl₃). ¹H NMR (300 MHz, CDCl₃): δ 1.39 (s, 3H, CH₃), 1.45 (s, 9H, COOtBu), 1.50-2.32 (10H, CH₂), 3.72, 3.76 (2 d, 2H, J=11.5 Hz, NH—CH₂-Ph), 4.47 (dd, 1H, J=7.8 Hz, J=5.7 Hz, CH—COOtBu), 4.56 (m, 1H, CO—N—CH), 7.20-7.40 (5H, Ph). ¹³C NMR (75.4 MHz, CDCl₃): δ 175.9, 171.6, 128.7, 128.3, 126.8, 80.7, 62.7, 61.4, 57.8, 47.7, 35.2, 34.9, 33.4, 29.7, 28.0, 26.8, 26.1, 22.4. FAB⁺MS: calc. C₂₂H₃₂N₂O₃ 372.24, found 373 [M+1]⁺. Elem. anal. calc. C₂₂H₃₂N₂O₃: C, 70.94; H, 8.66; N, 7.52; found C, 71.10; H, 8.44; N, 7.45.

Lactam 12: [α]_{D}²²=−50.8 (c=1.05, CHCl₃). ¹H NMR (400 MHz, CDCl₃): δ 1.39 (s, 3H, CH₃), 1.47 (s, 9H, COOtBu), 1.68-2.02 (8H, CH₂), 2.16, 2.29 (2 m, 2H, CH₂), 2.57 (sb, 1H, NH), 3.69, 3.75 (2 d, 2H, J=11.4 Hz, NH—CH₂-Ph), 4.00 (m, 1H, CO—N—CH), 4.55 (dd, 1H, J=8.2 Hz, J=4.5 Hz, CH—COOtBu), 7.20-7.48 (5H, Ph). ¹³C NMR (50.3 MHz, CDCl₃): δ 175.9, 171.8, 141.2, 128.8, 128.4, 126.9, 81.1, 63.4, 62.7, 58.4, 48.6, 35.3, 34.5, 33.3, 29.9, 28.2, 26.6, 24.7, 23.5. FAB⁺MS: calc. C₂₂H₃₂N₂O₃ 372.24, found 373 [M+1]⁺. Elem. anal. calc. C₂₂H₃₂N₂O₃: C, 70.94; H, 8.66; N, 7.52; found C 70.88, H 8.60, N 7.59.

Lactam 13: [α]_{D}²²=−114.7 (c=1.02, CHCl₃). ¹H NMR (300 MHz, CDCl₃): δ 1.48 (s, 9H, COOtBu), 1.53-2.30 (8H), 2.51 (m, 1H, CHN), 2.85, 3.06 (2 d, 2H, J=12.6 Hz, PhCH₂C), 3.80 (s, 2H, PhCH₂NH), 4.24 (dd, 1H, J=7.2 Hz, J=1.7 Hz, CHCOOtBu), 7.15-7.43 (10H, Ph). ¹³C NMR (50.3 MHz, CDCl₃): δ 172.7, 171.7, 140.6, 136.7, 130.9, 128.9, 128.5, 128.4, 128.3, 128.0, 127.9, 127.1, 126.9, 81.4, 61.9, 59.9, 59.7, 49.1, 47.1, 31.5, 30.2, 29.9, 28.6, 28.4, 28.2, 28.1. FAB⁺MS: calc. C₂₇H₃₄N₂O₃ 434.26, found 435 [M+1]⁺. Elem. anal. calc. C₂₇H₃₄N₂O₃: C, 74.62; H, 7.89; N, 6.45; found C 74.47, H 7.75, N 6.57.

Lactam 14: pf=161-163° C. [α]_{D}²²=−35.5 (c=1.06, CHCl₃). ¹H NMR (300 MHz, CDCl₃): δ 1.51 (s, 9H, COOtBu), 1.70-2.13 (8H), 2.98, 3.10 (2 d, 2H, J=14.0 Hz, PhCH₂C), 3.57 (m, 1H, CHN), 3.61, 3.68 (2 d, 2H, J=12.5 Hz, PhCH₂NH), 4.35 (dd, 1H, J=9.0 Hz, J<1 Hz, CHCOOtBu), 7.20-7.33 (10H, Ph). ¹³C NMR (75.4 MHz, CDCl₃): δ 173.1, 171.4, 140.8, 136.8, 131.2, 130.8, 128.7, 128.2, 128.1, 127.8, 126.7, 126.4, 81.1, 62.2, 60.5, 59.7, 59.0, 48.0, 44.6, 31.8, 29.7, 28.8, 28.6, 28.3, 28.0, 26. FAB⁺MS: calc. C₂₇H₃₄N₂O₃ 434.26, found 435 [M+1]⁺. Elem. anal. calc. C₂₇H₃₄N₂O₃: C 74.62, H 7.89, N 6.45; found C, 74.67; H, 7.95; N, 6.28.

Lactam 15: [α]_{D}²²=−68.7 (c=0.64, CHCl₃). ¹H NMR (400 MHz, CDCl₃): δ 1.50 (s, 9H, COOtBu), 1.76 (m, 1H), 1.93-2.24 (7H), 2.39 (m, 2H, CH₂CH═CH₂), 3.51 (m, 1H, CHN), 3.72, 3.78 (2 d, 2H, J=11.1 Hz, PhCH₂NH), 4.36 (dd, 1H, J=8.8 Hz, J<1 Hz, CHCOOtBu), 5.14 (m, 2H, CH═CH₂), 5.78 (m, 1H, CH═CH₂), 7.20-7.40 (5H, Ph). ¹³C NMR (50.3 MHz, CDCl₃): δ 171.8, 134.2, 133.5, 128.9, 128.5, 127.0, 119.2, 81.4, 60.6, 60.4, 60.0, 49.0, 48.1, 45.7, 44.2, 31.8, 30.2, 29.9, 28.7, 28.6, 28.1, 26.8. FAB⁺MS: calc. C₂₃H₃₂N₂O₃ 384.24, found 385 [M+1]⁺. Elem. anal. calc. C₂₃H₃₂N₂O₃: C, 71.84; H, 8.39; N, 7.29; found C, 71.72; H, 8.23; N, 7.46.

Lactam 16: [α]_{D}²²=−42.9 (c=1.07, CHCl₃). ¹H NMR (400 MHz, CDCl₃): δ 1.50 (s, 9H, COOtBu), 1.69-1.85 (2H), 1.94-2.06 (5H), 2.12 (m, 1H), 2.54 (m, 2H, CH₂CH═CH₂), 3.59 (m, 1H, CHN), 3.62, 3.70 (2 d, 2H, J=12.2 Hz, PhCH₂NH), 4.37 (dd, 1H, J=9.4 Hz, J<1 Hz, CHCOOtBu), 5.11 (m, 2H, CH═CH₂), 6.00 (m, 1H, CH═CH₂), 7.20-7.40 (5H, Ph). ¹³C NMR (75.4 MHz, CDCl₃): δ 171.3, 133.6, 129.5, 129.0, 128.7, 128.6, 127.4, 119.0, 81.5, 61.5, 60.7, 60.3, 59.3, 52.3, 48.0, 43.9, 31.9, 29.9, 28.7, 28.1, 26.7. FAB⁺MS: calc. C₂₃H₃₂N₂O₃ 384.24, found 385 [M+1]⁺. Elem. anal. calc. C₂₃H₃₂N₂O₃: C, 71.84; H, 8.39; N, 7.29; found C, 71.95; H, 8.29; N, 7.39.

Lactam 17: ¹H NMR (200 MHz, CDCl₃): δ 1.49 (s, 9H, COOtBu), 1.53-2.25 (10H), 3.22, 3.83 (2 d, 2H, J=14.0 Hz, PhCH₂C), 3.98, 4.05 (2 d, 2H, J=11.9 Hz, PhCH₂NH), 4.30 (m, 1H, CHN), 4.47 (m, 1H, CHCOOtBu), 7.13-7.45 (10H, Ph). ¹³C NMR (50.3 MHz, CDCl₃): δ 174.8, 171.6, 142.0, 138.6, 131.9, 129.9, 128.8, 127.7, 127.6, 127.5, 127.4, 127.3, 127.2, 127.0, 126.1, 125.3, 81.5, 64.2, 62.4, 57.3, 47.5, 40.2, 35.9, 33.9, 32.7, 29.8, 28.2, 26.5, 23.0. FAB⁺MS: calc. C₂₈H₃₆N₂O₃ 448.27, found 449 [M+1]⁺. Elem. anal. calc. C₂₈H₃₆N₂O₃: C, 74.97; H, 8.09; N, 6.24; found C, 74.77; H, 8.01; N, 6.39.

Lactam 18: ¹H NMR (200 MHz, CDCl₃): δ 1.51 (s, 9H, COOtBu), 1.60-2.41 (10H), 3.10, 3.65 (2 d, 2H, J=13.9 Hz, PhCH₂C), 3.71, 3.79 (2 d, 2H, J=12.0 Hz, PhCH₂NH), 4.18 (m, 1H, CHN), 4.65 (m, 1H, CHCOOtBu), 7.20-7.48 (10H, Ph). ¹³C NMR (50.3 MHz, CDCl₃): δ 174.0, 172.0, 141.5, 136.3, 131.0, 128.4, 128.2, 127.9, 126.0, 81.3, 65.5, 62.0, 57.2, 48.2, 44.8, 34.9, 32.3, 32.0, 28.2, 26.3, 22.5. FAB⁺MS: calc. C₂₈H₃₆N₂O₃ 448.27, found 449 [M+1]⁺. Elem. anal. calc. C₂₈H₃₆N₂O₃: C, 74.97; H, 8.09; N, 6.24; found C 75.02, H 8.15, N 6.10.

Lactam 19: ¹H NMR (200 MHz, CDCl₃): δ 1.45 (s, 9H, COOtBu), 1.48-2.80 (12H), 3.75, 3.82 (2 d, 2H, J=12.1 Hz, NH—CH₂-Ph), 4.39 (m, 1H, CHN), 4.62 (m, 1H, CHCOOtBu), 5.21 (m, 2H, CH₂—CH═CH₂), 5.89 (m, 1H, CH₂—CH═CH₂), 7.15-7.42 (5H, Ph). ¹³C NMR (50.3 MHz, CDCl₃): δ 174.0, 171.4, 141.3, 134.6, 128.5, 128.3, 126.9, 118.8, 111.1, 80.2, 67.2, 62.6, 63.5, 59.5, 58.5, 47.3, 44.6, 41.3, 35.4, 33.0, 29.8, 28.0, 26.6, 22.2. FAB⁺MS: calc. C₂₄H₃₄N₂O₃ 398.26, found 399 [M+1]⁺. Elem. anal. calc. C₂₄H₃₄N₂O₃: C 72.33, H 8.60, N 7.03; found C, 72.28; H, 8.74; N, 7.19.

Lactam 20: ¹H NMR (200 MHz, CDCl₃): δ 1.49 (s, 9H, COOtBu), 1.58-2.68 (12H), 3.58, 3.69 (2 d, 2H, J=11.8 Hz, NH—CH₂-Ph), 4.15 (m, 1H, CHN), 4.58 (m, 1H, CHCOOtBu), 5.10 (m, 2H, CH₂—CH═CH₂), 5.82 (m, 1H, CH₂—CH═CH₂), 7.20-7.45 (5H, Ph). ¹³C NMR (50.3 MHz, CDCl₃): δ 172.0, 134.9, 128.3, 128.2, 128.1, 126.9, 118.8, 81.0, 62.9, 57.9, 49.2, 48.8, 35.6, 34.8, is 33.0, 32.0, 30.0, 28.0, 26.4, 22.2. FAB⁺MS: calc. C₂₄H₃₄N₂O₃ 398.26, found 399 [M+1]⁺. Elem. anal. calc. C₂₄H₃₄N₂O₃: C, 72.33; H, 8.60; N, 7.03; found C, 72.42; H, 8.79; N, 6.86.

### EXAMPLE 2

General Procedure E: Synthesis of Cyclic Peptides Containing the Sequence RGD of the General Formula (II).

The bicyclic lactams of the general formula (I) (1 mmol) were treated at room temperature with a mixture of CF₃COOH (3.8 ml) and CH₂Cl₂ (10 ml) to remove the tert-butyl group. After evaporation, the residue was treated with anhydrous THF (6 ml), to which there was added 4-methyl morpholine (0.55 ml). To the solution cooled to −30° C. there was slowly added isobutyl chloroformiate (0.17 ml). Then, to the suspension stirred for 30 minutes at −30° C., there was then added a solution of H-Arg(Pmc)-Gly-OMe (1.29 g) in anhydrous THF (4 ml). The mixture was left to warm up to room temperature and left at this temperature overnight. After filtration on Celite to eliminate the insoluble salts, the crude product was purified by flash chromatography to obtain the pseudotetrapeptides (88-98% in 2 passages). The pseudotetrapeptides (1 mmol) were dissolved in MeOH (10 ml) and hydrogenated at atmospheric pressure using a catalytic amount of 10% Pd/C to eliminate the N-a benzyl group. The catalyst was removed by means of filtration on Celite to obtain, after evaporation at reduced pressure, the corresponding amines. To the solution of Z-Asp(tBu)—OH (648 mg) in anhydrous THF (10 ml), there was added 4-methyl morpholine (0.77 ml) and, slowly at −30° C., isobutyl chloroformiate (0.29 ml). After 30 minutes at this temperature there was added a solution of amine (1 mmol) in anhydrous THF (10 ml), and the mixture was slowly brought to room temperature and stirred overnight. The insoluble salts were removed by filtration on Celite, and after evaporation the residue was purified by flash chromatography to obtain the pseudopentapeptides (or peptidomimetic derivatives) (71-88% in 2 passages). To the solution of these peptides (1 mmol) in anhydrous THF (10 ml) there was added benzyl alcohol (10.3 ml), molecular sieves (2 g), Ti(OiPr)₄ (0.07 ml), and the mixture was heated to boiling for 5 days. The insoluble residues were eliminated by filtration on Celite, and after evaporation of the solvent the residue was recovered with CH₂Cl₂, washed with HCl 2N, and purified by flash chromatography to obtain the pseudopentapeptides (79-94%). The hydrogenation of the pseudopentapeptides (1 mmol) in MeOH (10 ml) with a catalytic amount of 10% Pd/C was necessary to remove the Cbz and benzyl groups simultaneously. After filtration on Celite to eliminate the catalyst and evaporation of the solvent, the deprotected pseudopentapeptides were dissolved in DMF (1000 ml), and the condensing system of Carpino [HATU (760 mg), HOAt (272 mg), 2,4,6-collidine (0.26 ml)] was used for cyclization. After 48-72 hours, the solvent was evaporated at reduced pressure; the residue was recovered with CH₂Cl₂, washed with saturated NaHCO₃ and KHSO₄ 1M. After evaporation the residue was purified by flash chromatography to obtain 22-25 cyclic pseudopentapeptides (64-78% in 2 passages). The deprotection of the side chains was obtained by treating the cyclic pseudopentapeptides (1 mmol) with CF₃COOH (330 ml) in the presence of ion scavengers. After evaporation the residue was dissolved in water and washed with iPr₂O. The purification of the crude products was conducted with Semi-preparative HPLC [column: SymmetryPrep C₁₈ 7 μm (7.8×300 mm—Waters)] using a gradient of 0-50% of MeCN in H₂O/0.1% CF₃COOH. The determination of the purity was conducted with analytical HPLC [column: Symmetry C₁₈ 5 μm (4.6×250 mm—Waters)] using the same gradient. The excess of CF₃COOH was removed in vacuum conditions, and treatment with gaseous HCl enabled conversion of the trifluoroacetate into chlorides, to obtain 26-29 (71-96% in 2 passages), ready for the biological assays.

Analytical data of the cyclic pseudopentapeptides (or peptidomimetic compounds):

Compound 22: pf=170-172° C. [α]_{D}²²=−42.1 (c=1.01, CHCl₃). ¹H NMR (400 MHz, CDCl₃): δ 1,31 (s, 6H, CH₃ Pmc), 1.50 (s, 9H, COOtBu), 1.52-2.25 (16H), 2.11, 2.58, 2.60 (3 s, 9H, CH₃ Pmc), 2.61 (m, 3H, CH₂ Pmc, CHHCOOtBu Asp), 2.97 (dd, 1H, J=17.1 Hz, J=4.4 Hz, CHHCOOtBu Asp), 3.28 (m, 4H, CHN, CHHPh, CH₂NHC═NH), 3.38 (m, 1H, CHH Gly), 3.60 (d, 1H, J=12.9 Hz, CHHPh), 3.91 (dd, 1H, J=14.0 Hz, J=5.7 Hz, CHH Gly), 4.12 (dd, 1H, J=7.7 Hz, J=7.7 Hz, CHCONH lactam), 4.63 (m, 1H, CHNH Arg), 4.77 (m, 1H, CHCH₂COOtBu Asp), 6.1-6.4 (3H, (NH)₂C═NH), 6.55 (d, 1H, J=7.9 Hz, NHArg), 7.0-7.3 (5H, Ph), 7.16 (s, 1H, NH lactam), 7.79 (dd, 1H, J=9.2 Hz, J, NH Asp), 8.25 (m, 1H, NH Gly). ¹³C NMR (50.3 MHz, CDCl₃): δ 174.0, 173.2, 171.6, 170.2, 169.8, 156.5, 153.7, 136.4, 135.7, 135.0, 130.4, 128.5, 127.2, 124.1, 118.1, 81.4, 73.8, 71.9, 71.3, 67.8, 66.0, 62.0, 52.4, 50.7, 45.6, 40.5, 35.6, 33.0, 31.9, 31.2, 30.0, 28.3, 27.0, 26.9, 25.5, 21.6, 19.5, 18.7, 18.4, 17.7, 12.3. FAB⁺MS: calc. C₄₇H₆₆N₈O₁₀S 934.46, found 935 [M+1]⁺. Elem. anal. calc. C₄₇H₆₆N₈O₁₀S: C 60.37, H 7.11, N 11.98; found C, 60.41; H, 7.21; N, 11.85.

Compound 23: pf=175-177° C. [α]_{D}²²=43.4 (c=1.03, CHCl₃). ¹H NMR (400 MHz, CDCl₃): δ 1,32 (s, 6H, CH₃ Pmc), 1.48 (s, 9H, COOtBu), 1.55-2.35 (14H), 2.12, 2.58, 2.60 (3S, 9H, CH₃ Pmc), 2.40-2.75 (6H, CH₂ Pmc, CH₂COOtBu Asp), 3.22 (m, 3H, CHH Gly, CH₂NHC═NH), 3.51 (d, 1H, J=14.2 Hz, CHHPh), 3.70 (m, 2H, CHHPh, CHH Gly), 4.19 (m, 1H, CHNH Arg), 4.35 (m, 2H, CHN, CHCONH lactam), 4.98 (m, 1H, CHCH₂COOtBu Asp), 6.05-6.5 (5H, (NH)₂C═NH, NH Arg, NH Asp), 7.10-7.35 (5H, Ph), 7.37 (m, 1H, NH Gly), 8.00 (s, 1H, NH lactam). ¹³C NMR (50.3 MHz, CDCl₃): δ 174.0, 171.6, 171.2, 170.0, 169.9, 136.5, 131.3, 128.6, 127.6, 124.3, 118.3, 81.6, 73.9, 66.4, 65.5, 59.7, 50.9, 45.9, 34.8, 34.5, 32.9, 29.9, 28.2, 27.0, 23.7, 21.6, 18.7, 17.7, 12.3. FAB⁺MS: calc. C₄₇H₆₆N₈O₁₀S 934.46, found 935 [M+1]⁺. Elem. anal. calc. C₄₇H₆₆N₈O₁₀S: C, 60.37; H, 7.11; N, 11.98; found C, 60.30; H, 7.09; N, 12.01.

Compound 24: pf=178-180° C. [α]_{D}²²=−42.2 (c=1.07, CHCl₃). ¹H NMR (400 MHz, CDCl₃): δ 0.60 (m, 1H), 1.12 (m, 1H), 1,32 (s, 6H, CH₃ Pmc), 1.38 (s, 9H, COOtBu), 1.50-2.30 (12H), 2.10, 2.57, 2.59 (3 s, 9H, CH₃ Pmc), 2.54 (m, 1H, CHHCOOtBu Asp), 2.64 (m, 3H, CH2 Pmc, CHHCOOtBu Asp), 2.86 (d, 1H, J=12.9 Hz, CHHPh), 3.22 (m, 1H, CHHNHC═NH), 3.34 (m, 3H, CHHPh, CHHNHC═NH, CHH Gly), 3.78 (m, 1H, CHN), 4.40 (dd, 1H, J=9.0 Hz, J=9.0 Hz, CHCONH lactam), 4.53 (dd, 1H, J=14.5 Hz, J=9.2 Hz, CHH Gly), 4.67 (m, 2H, CHNH Arg, CHCH₂COOtBu Asp), 6.1-6.4 (3H, (NH)₂C═NH), 6.68 (m, 1H, NHAsp), 7.01 (s, 1H, NH lactam), 7.10-7.40 (5H, Ph), 7.24 (m, 1H, NH Arg), 7.77 (m, 1H, NH Gly). ¹³C NMR (50.3 MHz, CDCl₃): δ 172.7, 171.7, 171.2, 170.9, 169.3, 156.4, 153.7, 135.7, 135.1, 133.5, 130.2, 129.0, 127.8, 124.1, 118.0, 81.8, 73.8, 66.0, 61.9, 59.8, 59.1, 52.0, 50.3, 44.8, 44.2, 40.9, 37.6, 33.2, 33.0, 30.8, 29.5, 28.6, 28.1, 27.0, 26.9, 26.8, 25.3, 21.6, 18.7, 17.6, 15.4, 12.3. FAB⁺MS: calc. C₄₆H₆₄N₈O₁₀S 920.45, found 921 [M+1]⁺. Elem. anal. calc. C₄₆H₆₄N₈O₁₀S: C, 59.98; H, 7.00; N, 12.17; found C 60.11, H 7.09, N 12.02.

Compound 25: pf=179-181° C. [α]_{D}²²=−16.8 (c=1.00, CHCl₃). ¹H NMR (400 MHz, CDCl₃): δ 1,33 (s, 15H, CH₃ Pmc, COOtBu), 1.38-2.50 (16H), 2.10, 2.57, 2.60 (3 s, 9H, CH₃ Pmc), 2.50-2.70 (4H, CH₂ Pmc, CH₂COOtBu Asp), 3.22 (m, 2H, CHHNHC═NH, CHHPh), 3.33 (m, 3H, CHHPh, CHHNHC═NH, CHH Gly), 4.41 (m, 1H, CHH Gly), 4.50 (m, 2H, CHNH Arg, CHN), 4.60 (m, 2H, CHCONH lactam, CHCH₂COOtBu Asp), 6.10-6.50 (3H, (NH)₂C═NH), 6.82 (s, 1H, NH lactam), 6.96 (m, 1H, NH Asp), 7.19 (d, 1H, J=6.6 Hz, NH Arg), 7.20-7.40 (5H, Ph), 7.74 (m, 1H, NH Gly). ¹³C NMR (50.3 MHz, CDCl₃): δ 173.3, 172.8, 171.4, 170.8, 169.7, 156.2, 135.8, 135.3, 130.8, 128.9, 127.5, 124.3, 118.2, 81.6, 73.9, 65.5, 64.2, 57.0, 50.4, 44.7, 41.6, 40.8, 36.7, 33.0, 32.7, 28.5, 28.1, 27.3, 27.0, 25.7, 21.6, 19.3, 18.7, 17.7, 12.3. FAB⁺MS: calc. C₄₇H₆₆N₈O₁₀S 934.46, found 935 [M+1]⁺. Elem. anal. calc. C₄₇H₆₆N₈O₁₀S: C 60.37, H 7.11, N 11.98; found C, 60.26; H, 7.03; N, 11.87.

Compound 26: purity HPLC: 98.2%. [α]_{D}²²=−85.9 (c=0.95, MeOH). ¹H NMR (400 MHz, D₂O): δ 1.5-2.2 (13H), 2.59 (m, 1H) 2.69, 2.90 (2 dd, 2H, J=5.9 Hz, J=7.8 Hz, J=17.0 Hz, CH₂COOH Asp), 3.15 (m, 2H, CH₂NHC═NH Arg), 3.23, 3.46 (2 d, 2H, J=13.7 Hz, PhCH₂), 3.50, 3.91 (2 m, 2H, CH₂ Gly), 4.01 (m, 1H, CHN), 4.22 (dd, 1H, J=8.0 Hz, J=8.0 Hz, CHCONH lactam), 4.31 (m, 1H, NHCHCH₂ Arg), 4.79 (m, 1H, CHCH₂COOH Asp), 6.85 (d, 1H, J=8.4 Hz, NH Arg), 7.0, 7.26 (2 m, 5H, Ph), 7.78 (s, 1H, NH lactam). ¹³C NMR (75.4 MHz, D₂O): δ 175.5, 174.3, 174.0, 172.4, 171.1, 136.5, 130.6, 129.2, 128.0, 67.8, 66.9, 59.5, 53.2, 50.9, 44.5, 41.1, 38.7, 34.2, 33.2, 30.8, 29.6, 27.1, 25.0, 22.3. FAB⁺MS: calc. C₂₉H₄₁ClN₈O₇ 648.28, found 613 [M—Cl]⁺. Elem. anal. calc. C₂₉H₄₁ClN₈O₇: C 53.66, H 6.37, N 17.26; found C, 53.78; H, 6.45; N, 17.38.

Compound 27: purity HPLC: 99.5%. [α]_{D}²²=−54.7 (c=1.01, MeOH). ¹H NMR (400 MHz, D₂O): δ 1,3-1.55 (3H), 1.65-2.10 (10H), 2.15 (m, 1H), 2.33-2.52 (3H), 2.74 (dd, 2H, J=6.8 Hz, J=17.0 Hz, CH₂COOH Asp), 3.17 (m, 2H, CH₂NHC═NH Arg), 3.53 (m, 3H, PhCH₂, CHH Gly), 3.68 (d, 1H, J=13.9 Hz, CHH Gly), 4.18 (dd, 1H, J=4.7 Hz, J=11.0 Hz, NHCHCH₂ Arg), 4.33 (m, 2H, CHN, CHCONH lactam), 4.88 (m, 1H, CHCH₂COOH Asp), 7.15, 7.32 (5H, Ph). ¹³C NMR (75.4 MHz, D₂O): δ 175.1, 174.3, 173.9, 171.5, 171.3, 136.9, 131.2, 129.3, 128.6, 109.4, 66.7, 60.3, 54.1, 53.3, 51.6, 45.7, 41.2, 36.0, 34.8, 33.7, 33.3, 28.2, 26.6, 25.5, 23.7. FAB⁺MS: calc. C₂₉H₄₁ClN₈O₇ 648.28, found 613 [M—Cl]⁺. Elem. anal. calc. C₂₉H₄₁ClN₈O₇: C, 53.66; H, 6.37; N, 17.26; found C, 53.51; H, 6.48; N, 17.13.

Compound 28: purity HPLC: 96.1%. [α]_{D}²²=−96.8 (c=1.03, MeOH). ¹H NMR (400 MHz, D₂O): δ 0.11 (m, 1H), 0.92 (m, 1H), 1.50 (m, 2H), 1.62 (m, 3H), 1.78-1.96 (2H), 2.09 (m, 2H), 2.47 (m, 1H), 2.68, 2.76 (2 dd, 2H, J=6.6 Hz, J=7.7 Hz, J=16.0 Hz, CH₂COOH Asp), 2.81 (d, 1H, J=12.7 Hz, PhCHH), 3.16 (m, 2H, CH₂NHC═NH Arg), 3.37 (2 d, 2H, J=12.9 Hz, J=14.5 Hz, PhCHH, CHH Gly), 3.60 (m, 1H, CHN), 4.28 (d, 1H, J=14.5 Hz, CHH Gly), 4.36 (dd, 1H, J=8.7 Hz, J=8.7 Hz, CHCONH lactam), 4.42 (dd, 1H, J=7.2 Hz, J=7.2 Hz, NHCHCH₂ Arg), 4.76 (m, 1H, CHCH₂COOH Asp), 7.00-7.20 (5H, Ph). ¹³C NMR (75.4 MHz, D₂O): δ 174.8, 173.5, 172.7, 172.3, 171.8, 135.7, 130.6, 129.6, 128.5, 62.2, 61.7, 60.6, 60.0, 53.6, 53.0, 50.2, 44.9, 44.5, 41.3, 36.9, 35.6, 33.2, 31.6, 29.8, 28.4, 26.7, 25.3, 25.0. FAB⁺MS: calc. C₂₈H₃₉ClN₈O₇ 634.26, found 599 [M—Cl]⁺. Elem. anal. calc. for C₂₈H₃₉ClN₈O₇: C, 52.95; H, 6.19; N, 17.64; found C 53.03, H 6.35, N 17.68.

Compound 29: purity HPLC: 97.5%. [α]_{D}²²=+38.1 (c=0.68, MeOH). ¹H NMR (400 MHz, D₂O): δ 1.40-1.89 (10H), 2.00-2.38 (4H) 2.81 (m, 2H, CH₂COOH Asp), 3.15 (m, 4H, CH₂NHC═NH Arg, PhCH₂), 3.46 (d, 1H, J=14.8 Hz, CHH Gly), 4.14 (m, 1H, CHN), 4.22 (m, 2H, NHCHCH₂ Arg, CHH Gly), 4.44 (m, 1H, CHCONH lactam), 4.62 (m, 1H, CHCH₂COOH Asp), 7.12, 7.31 (2 m, 5H, Ph). ¹³C NMR (75.4 MHz, D₂O): δ 175.6, 175.1, 173.8, 173.4, 171.6, 136.8, 131.5, 129.1, 127.9, 65.7, 64.9, 59.8, 54.3, 51.0, 44.8, 41.2, 35.0, 33.4, 32.5, 29.3, 28.0, 27.6, 25.1, 21.4. FAB⁺MS: calc. C₂₉H₄₁ClN₈O₇ 648.28, found 613 [M—Cl]⁺. Elem. anal. calc. C₂₉H₄₁ClN₈O₇: C, 53.66; H, 6.37; N, 17.26; found C, 53.50; H, 6.47; N, 17.22.

1. The compounds having the following the general formula: 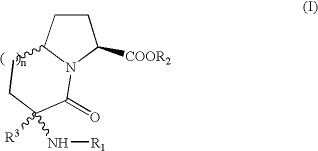
where:
R₁ is chosen from hydrogen, a lower alkyl, and a suitable protective group of the amine;
R₂ is chosen between hydrogen, and a suitable protective group of the carboxyl;
R₃ is chosen from a benzyl, substituted benzyl, allyl, hydroxypropyl, hydroxyethyl, and lower alkyl;
n is a number chosen from 0, 1, 2;
including the salts, the racemates, the individual enantiomeric forms, the individual diastereoisomeric forms, or their mixtures.

2. The compounds according to claim 1, characterized in that said lower alkyl is a C₁-C₄ alkyl group.

3. The compounds according to claim 1, characterized in that said suitable protective group is chosen between an alkyl ester and a benzyl ester.

4. The compounds according to claim 1, characterized in that n is chosen equal to 1, and R₃ is chosen as a benzyl.

5. The compounds according to claim 1, characterized in that n is chosen equal to 1, and R₃ is chosen as an allyl.

6. The compounds according to claim 1, characterized in that n is chosen equal to 2, and R₃ is chosen as a benzyl.

7. The compounds according to claim 1, characterized in that n is chosen equal to 2, and R₃ is chosen as an allyl.

8. The compounds according to claim 1, characterized in that n is chosen equal to 2, and R₃ is chosen as a methyl.

9. A process for the preparation of the compounds according to claim 1, which comprises the following steps:
formation, in suitable reaction conditions, of a carbanion in position 3 starting from the compound (Ia) having the following formula: 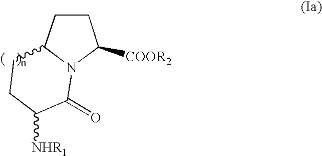
or by one of its suitable derivatives,
alkylation of said carbanion to obtain the compound of the general formula (I)
including the salts, the racemates, the individual enantiomeric forms, the individual diastereoisomeric forms, or their mixtures.

10. A process according to claim 9, characterized in that:
R₁ is chosen from hydrogen, a lower alkyl, and a suitable protective group of the amine;
R₂ is chosen between hydrogen, and a suitable protective group of the carboxyl;
R₃ is chosen from benzyl, substituted benzyl, allyl, hydroxypropyl, hydroxyethyl, lower alkyl;
n is a number chosen from 0, 1, 2;

11. The process according to claim 10, characterized in that said lower alkyl is a C₁-C₄ alkyl group.

12. The process according to claim 9, characterized in that said R₃ is chosen as an allyl.

13. The process according to claim 12, characterized in that said allyl is converted into a hydroxyethyl or a hydroxypropyl.

14. Use of the compounds according to claim 1 as intermediates in the synthesis of peptidomimetic compounds.

15. Use according to claim 14 in the synthesis of peptidomimetic compounds comprising the sequence RGD (Arg-Gly-Asp).

16. Peptidomimetic compounds comprising the sequence RGD (Arg-Gly-Asp) (Arginine, Glycine, Aspartic acid) having the following general formula (II): 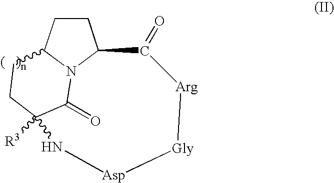
where:
R₃ is chosen from benzyl, substituted benzyl, allyl, hydroxypropyl, hydroxyethyl, lower alkyl;
n is a number chosen from 0, 1, 2;
including the salts, the racemates, the individual enantiomeric forms, the individual diastereoisomeric forms, or their mixtures.

17. The compounds according to claim 16, characterized in that said lower alkyl is a C₁-C₄ alkyl group.

18. Compound according to claim 16, characterized in that n is chosen equal to 1 and R₃ is chosen as a benzyl.

19. Compound according to claim 16, characterized in that n is chosen equal to 2 and R₃ is chosen as a benzyl.

20. The compounds according to claim 16, characterized in that said R₃ is an allyl.

21. The compounds according to claim 16, characterized in that said R₃ is hydroxyethyl or hydroxypropyl.

22. The process for the preparation of compounds according to claim 16, which comprises the following steps:
reaction of chemoselective deprotection of the carboxylic group of the compound of the general formula (I) according to claim 1 and condensation with the dipeptide Arg-Gly appropriately protected and previously prepared;
reaction of chemoselective protection of the amine group of the azabicycloalkane and subsequent condensation with appropriately protected aspartic acid;
conversion of glycine by means of transesterification reaction followed by the simultaneous removal of the protective group of glycine and aspartic acid;
intramolecular cyclization mediated by condensing agents and subsequent deprotection of the protective groups of the side chains of amino acids.

23. The process according to claim 22, characterized in that said deprotection of the amine group of the azabicycloalkane is obtained by means of catalytic hydrogenation.

24. The process according to claim 22, characterized in that said conversion of glycine is obtained by transesterification of the methyl ester in benzyl ester and in that said subsequent removal of the protective group of glycine and aspartic acid is obtained by catalytic hydrogenation.

25. Use of the compounds according to claim 16 as inhibitors of integrines.

26. Use according to claim 25 for the inhibition of αvβ3 and αvβ5 integrines.

27. Use of the compounds according to claim 16 as drugs for inhibiting angiogenesis.

28. Use of the compounds according to claim 16 as drugs in the treatment of pathological conditions of a tumoral origin, in metastasized tumoral processes, retinopathies, acute renal damage and osteoporosis.

29. Use of the compounds according to claim 16 as “reverse-turn” inducers.

30. Use of the compounds according to claim 16 as mediators for the transport and release of drugs.

31. Pharmaceutical compositions that comprise at least one compound according to claim 16 in a mixture with vehicles and/or excipients which are acceptable from the pharmaceutical point of view.

32. Use of the pharmaceutical compositions according to claim 31 as inhibitors of integrines.

33. Use of the pharmaceutical compositions according to claim 31 for the inhibition of αvβ3 and αvβ5 integrines.

34. Use of the pharmaceutical compositions according to claim 31 as angiogenesis inhibitors.

35. Use of the pharmaceutical compositions according to claim 31 in the treatment of pathological conditions of a tumoral origin, in metastasized tumoral processes, retinopathies, acute renal damage and osteoporosis.

36. Use of the pharmaceutical compositions according to claim 31 as mediators for the transport and release of drugs.
